(19) United States
(12) Patent Application Publication
(10) Pub. No.: US 2026/0078157 A1
(43) Pub. Date: Mar. 19, 2026

(54) FGF21 Mutant Polypeptides

(71) Applicant: AKERO THERAPEUTICS, INC., South San Francisco, CA (US)

(72) Inventors: Timothy P. ROLPH, Brockline, MA (US); Erik J. TILLMAN, Arlington, MA (US)

(21) Appl. No.: 19/110,673

(22) PCT Filed: Sep. 11, 2023
(86) PCT No.: PCT/US2023/073867

§ 371 Date: Mar. 11, 2025

Related U.S. Application Data
(60) Provisional application No. 63/405,758, filed on Sep. 12, 2022.

(51) Int. Cl. C07K 14/50; A61K 38/00; C07K 19/00

(52) U.S. Cl. CPC C07K 14/50; C07K 19/00; A61K 38/00; C07K 2319/30

(57) ABSTRACT

The disclosure provides a complex comprising (a) a first monomer comprising an Fc domain fused to two FGF21 domains comprising the amino acid sequence of SEQ ID NO: 2 or SEQ ID NO: 4, which may be the same or different, and (b) a second monomer comprising an Fc domain fused to at least one FGF21 domain comprising the amino acid sequence of SEQ ID NO: 2 or SEQ ID NO: 4, wherein at least two of the FGF21 domains of the complex comprise a free beta-klotho binding region.

### CROSS-REFERENCE TO RELATED APPLICATIONS

This application claims priority to U.S. Provisional Patent Application No. 63/405,758, filed Sep. 12, 2022, which is hereby incorporated by reference in its entirety.

### FIELD

The disclosure is related to polypeptides and complexes comprising multiple, mutant FGF21 domains.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 100,787 byte XML file named “58224_Seqlisting.xml”; created on Sep. 11, 2023.

### BACKGROUND

Fibroblast growth factor 21 (FGF21) is an endocrine hormone that acts on the liver, pancreas, muscle, and adipose tissue to regulate the metabolism of lipids, carbohydrates, and proteins. Acting as a paracrine hormone, human FGF21 also plays a critical role in protecting cells against stress. There is growing evidence that metabolic disease states like type 2 diabetes (T2D) and NASH are associated with resistance to endogenous FGF21, i.e., FGF21 is less effective in maintaining insulin sensitivity and protecting hepatocytes against multiple cellular stresses associated with high intracellular fat. The level of FGF21 appears higher in patients with metabolic disease, but it does not appear to be associated with improved FGF21 signaling. This may be associated with lower expression levels of FGF21's receptors. Genetic loss of function mutations in one or both of FGF21's receptors, beta-klotho (KLB) and FGFR1c, are associated with resistance to endogenous FGF21, including increased hepatic fibrinogenesis. Very rare cases carrying heterozygous LOF mutations in both KLB and FGFR1c, have profound resistance to FGF21 and to insulin, with early onset T2D and liver steatosis. Stone et al. Digenic Variants in the FGF21 Signaling Pathway Associated with Severe Insulin Resistance and Pseudoacromegaly. J Endocr Soc 4, bvaa138 (2020).

FGF21's attributes make FGF21 agonism a compelling therapeutic mechanism, but native FGF21 is limited by its short half-life in the bloodstream, and some studies have shown that beneficial effects associated with FGF21 administration diminish over longer treatment periods. For example, there is evidence with the single chain FGF21 analog, pegbelfermin, that the pharmacological response (adiponectin induction) and markers of efficacy wane with longer periods of treatment (e.g., 24 weeks) compared to biological responses achieved earlier in treatment periods (e.g., 8-12 weeks). Sanyal et al., AASLD The Liver Meeting 2021, Poster LP-40.

### SUMMARY

The disclosure provides a complex comprising (a) a first monomer comprising an Fc domain fused to two FGF21 domains comprising the amino acid sequence of SEQ ID NO: 2 or SEQ ID NO: 4, which may be the same or different, and (b) a second monomer comprising an Fc domain fused to at least one FGF21 domain comprising the amino acid sequence of SEQ ID NO: 2 or SEQ ID NO: 4, wherein at least two of the FGF21 domains of the complex comprise a free beta-klotho binding region. Optionally, (a) the first monomer comprises the amino acid sequence of SEQ ID NO: 4 fused to the N-terminus of the Fc domain and the amino acid sequence of SEQ ID NO: 2 fused to the C-terminus of the Fc domain. In other aspects, (a) the first monomer comprises one or more FGF21 domains of the amino acid sequence of SEQ ID NO: 2 fused to the N-terminus of the Fc domain (optionally via a linker) followed by another FGF21 domain comprising the amino acid sequence of SEQ ID NO: 2. In various aspects, (b) the second monomer comprises the amino acid sequence of SEQ ID NO: 4 fused to the N-terminus of the Fc domain and the amino acid sequence of SEQ ID NO: 2 fused to the C-terminus of the Fc domain. Alternatively, (b) the second monomer comprises the amino acid sequence of SEQ ID NO: 4 fused to the N-terminus of the Fc domain and the amino acid sequence of SEQ ID NO: 4 fused to the C-terminus of the Fc domain. In some aspects, (a) the first monomer comprises the amino acid sequence of SEQ ID NO: 2 fused to the N-terminus of the Fc domain and the amino acid sequence of SEQ ID NO: 2 fused to the C-terminus of the Fc domain; optionally, (b) the second monomer comprises the amino acid sequence of SEQ ID NO: 2 fused to the N-terminus of the Fc domain and the amino acid sequence of SEQ ID NO: 2 fused to the C-terminus of the Fc domain. The disclosure also provides a complex comprising two monomers comprising the amino acid sequence of SEQ ID NO: 18, two monomers comprising the amino acid sequence of SEQ ID NO: 19, or two monomers comprising the amino acid sequence of SEQ ID NO: 20.

A complex comprising (a) a first monomer comprising an Fc domain fused at the C-terminus with an FGF21 domain comprising the amino acid sequence of SEQ ID NO: 4 and (b) a second monomer comprising an Fc domain fused at the N-terminus with an FGF21 domain comprising the amino acid sequence of SEQ ID NO: 2 also is provided.

The disclosure also provides a peptide comprising an Fc domain fused with an FGF21 domain comprising the amino acid sequence of SEQ ID NO: 4 at the N-terminus and an FGF21 domain comprising the amino acid sequence of SEQ ID NO: 2 at the C-terminus, as well as a peptide comprising the amino acid sequence of SEQ ID NO: 4 fused to the amino acid sequence of SEQ ID NO: 2 via a linker (e.g., a linker comprising the amino acid sequence of SEQ ID NO: 9 and/or the amino acid sequence of SEQ ID NO: 10).

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. In addition, the invention includes, as an additional aspect, all aspects of the invention narrower in scope in any way than the variations specifically mentioned above.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. The terms “comprising,” “having,” “including,” and “containing” are to be construed as open-ended terms unless otherwise noted. If aspects of the invention are described as “comprising” a feature, aspects also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities should be understood as modified in all instances by the term “about” as that term would be interpreted by the person skilled in the relevant art. With respect to aspects of the invention described or claimed with “a,” “the,” or “an,” it should be understood that these terms mean “one or more” unless context unambiguously requires a more restricted meaning. With respect to elements described as one or more within a set, it should be understood that all combinations within the set are contemplated.

It should also be understood that when describing a range of values, the disclosure contemplates individual values found within the range. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional aspects that also are intended as aspects of the invention, irrespective of whether the combination of features is specified as an aspect of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention. The use of section headings, when present, is merely for the convenience of reading; it should be understood that all combinations of features described herein are contemplated.

### BRIEF DESCRIPTION OF THE DRAWINGS

FIGS. 1A-1O are schematics of representative mutant FGF21-based complexes and peptides of the disclosure. The lines and rectangles represent Fc domains, while the oval shapes represent FGF21 domains. Ovals comprising an elongated terminus represent FGF21 domains comprising a free beta-klotho binding region.

FIG. 2 provides various amino acid sequences described in the disclosure. The amino acid sequences are shown in N- to C-orientation. Plain text corresponds to Fc domain regions. Underlined text corresponds to linker sequences. Italicized text represents FGF21 domains.

FIG. 3 is a graph illustrating results of a cell-based potency assay performed in HEK293 cells overexpressing human KLB and FGFR1c, measuring Elk1 in response to FGF21 analogues signaling through a luciferase reporter system. EFX is denoted by circles; FGF21 comprising the modifications L98R, P171G, and A180E relative to mature, human FGF21 is denoted as squares; and ¾ EFX described herein is denoted as triangles.

### DETAILED DESCRIPTION

The disclosure provides polypeptide complexes and peptides comprising at least two mutant FGF21 domains, and in some instances at least three (e.g., four) mutant FGF21 domains. The mutant FGF21 domains include three modifications introduced into the native FGF21 sequence at L125R, P198G, and A207E, (corresponding to L98R, P171G, and A180E relative to mature, human FGF21). These modifications 1) decrease susceptibility to in vivo proteolytic degradation, 2) increase affinity for beta-klotho, and 3) decrease the propensity to aggregate (Hecht et al., PLOS One 2012; 7(11):e49345; Stanislaus et al., Endocrinology. 2017; 158(5):1314-1327). For the sake of clarity, the invention described herein does not encompass Efruxifermin (EFX). EFX is a 92.1 kDa, long-acting fibroblast growth factor 21 (FGF21) analogue comprising monomers which are fusion proteins comprising a human immunoglobulin IgG1 Fc fragment fused to a mutant FGF21 domain comprising modifications at positions L98R, P171G, and A180E (relative to mature, human FGF21). Each EFX molecule is a homodimer comprising a dimerized Fc domain wherein each Fc domain is fused to a single modified FGF21 polypeptide chain. EFX has 8 disulfide bonds, 6 intra-chain and 2 inter-chain. Two of the intrachain disulfide bonds are in the FGF21 polypeptide between Cys318 and Cys336, one for each monomer. EFX comprises the amino acid sequence set forth in SEQ ID NO: 1. EFX (and the various components which make up the molecule) have been further described in U.S. Pat. Nos. 8,034,770; 8,410,051; 8,642,546; 8,361,963; 9,273,106; 10,011,642; 8,188,040; 8,835,385; 8,795,985; 8,618,053; and 11,072,640; or International Patent Publication Nos. WO2009149171 and WO2010129503, the disclosures of which are incorporated herein by reference in their entireties.

In various aspects, the disclosure provides a complex comprising (a) a first monomer comprising an Fc domain fused to two or more FGF21 domains (e.g. two FGF21 domains) comprising the amino acid sequence of SEQ ID NO: 2 (or SEQ ID NO: 63 or 65) or SEQ ID NO: 4 (or SEQ ID NO: 64 or 66), which may be the same or different, and (b) a second monomer comprising an Fc domain fused to at least one FGF21 domain comprising the amino acid sequence of SEQ ID NO: 2 (or SEQ ID NO: 63 or 65) or SEQ ID NO: 4 (or SEQ ID NO: 64 or 66), wherein at least two of the FGF21 domains of the complex comprise a free beta-klotho binding region. The first and the second monomer associate via the Fc domains to generate a complex. The configuration of the first monomer is such that an FGF21 domain is fused to the N-terminus of the Fc domain and another FGF21 domain is fused to the C-terminus of the Fc domain (i.e., the Fc domain is flanked by two FGF21 domains, which may be the same or different, and are selected from FGF21 domains comprising the amino acid sequence of SEQ ID NO: 2, SEQ ID NO: 4, or SEQ ID NO: 63-66). The configuration of the complex is such that at least two of the FGF21 domains of the complex comprise a free beta-klotho binding region. A “beta-klotho binding region” of the FGF21 domain refers to the portion of the domain which binds beta-klotho; the portion is located at the terminus of the domain which comprises the amino acid sequence QGRSPSYES (SEQ ID NO: 3) found within the sequence of SEQ ID NO: 2 (or SEQ ID NO: 63 or 65) or the amino acid sequence SEYSPSRGQ (SEQ ID NO: 6) found within SEQ ID NO: 4 (or SEQ ID NO: 64 or 66). A “free beta-klotho binding region” refers to a configuration in which the beta-klotho binding region of the FGF21 domain is exposed and available to bind beta-klotho, i.e., the terminus of the FGF21 domain which comprises the beta-klotho region is not fused to the Fc domain or another FGF21 domain. In various aspects, the first monomer comprises one or more FGF21 domains comprising the amino acid sequence of SEQ ID NO: 2 fused to the N-terminus of the Fc domain (optionally via a linker) followed by another FGF21 domain comprising the amino acid sequence of SEQ ID NO: 2. Thus, the disclosure contemplates a structure wherein two, three, or four FGF21 domains of the amino acid sequence of SEQ ID NO: 2 are present at the N-terminus, in tandem, and fused to the N-terminus of the Fc domain, which is followed by one or more (e.g., one) FGF21 domain comprising SEQ ID NO: 2. Fusion to the Fc domain may be via a linker, although this is optional.

The disclosure contemplates aspects wherein the FGF21 domains comprise an amino acid sequence at least about 90% identical (e.g., at least about 91%, at least about 92%, at least about 93%, at least about 94%, at least about 95%, at least about 96%, at least about 97%, at least about 98%, at least about 99% or 100%) identical to SEQ ID NO: 2 or 4. Indeed, at least one natural isoform of FGF21 has been reported, wherein the proline at amino acid position 146 in the mature FGF21 protein is substituted with a leucine. The two isoforms are often referenced as the “P-form” (having a proline at position 146) and the “L-form” (having a leucine at position 146). In this regard, the disclosure includes FGF21 domains comprising the amino acid sequence of SEQ ID NO: 63 or SEQ ID NO: 64, wherein X is proline (e.g., SEQ ID NOs: 2 or 4, respectively) or leucine (e.g., SEQ ID NOs: 65 and 66, respectively). In instances where the FGF21 domains are less than 100% identical to SEQ ID NOs: 2, 4, or 63-66, the FGF21 domains retain at least the glutamic acid at position 180 relative to mature, human FGF21, and also optionally retain the arginine at position 98 and the glycine at position 171 set forth in SEQ ID NOs: 2, 4, and 63-66. Disclosure herein relating to SEQ ID NOs: 2 and 4 also applies to FGF21 domains comprising the amino acid sequence of any one of SEQ ID NOs: 63-66. For example the disclosure provides a complex comprising (a) a first monomer comprising an Fc domain fused to two FGF21 domains comprising the amino acid sequence of SEQ ID NO: 63 or SEQ ID NO: 64, which may be the same or different, and (b) a second monomer comprising an Fc domain fused to at least one FGF21 domain comprising the amino acid sequence of SEQ ID NO: 63 or SEQ ID NO: 64, wherein at least two of the FGF21 domains of the complex comprise a free beta-klotho binding region.

For example, the first monomer optionally comprises the amino acid sequence of SEQ ID NO: 4 (or SEQ ID NO: 64 or 66) fused to the N-terminus of the Fc domain and the amino acid sequence of SEQ ID NO: 2 (or SEQ ID NO: 63 or 65) fused to the C-terminus of the Fc domain. In various aspects, the second monomer comprises (i) the amino acid sequence of SEQ ID NO: 4 (or SEQ ID NO: 64 or 66) fused to the N-terminus of the Fc domain, (ii) the amino acid sequence of SEQ ID NO: 2 (or SEQ ID NO: 63 or 65) fused to the N-terminus of the Fc domain, (iii) the amino acid sequence of SEQ ID NO: 4 (or SEQ ID NO: 64 or 66) fused to the C-terminus of the Fc domain, or (iv) the amino acid sequence of SEQ ID NO: 2 (or SEQ ID NO: 63 or 65) fused to the C-terminus of the Fc domain. In various aspects, the second monomer comprises the amino acid sequence of SEQ ID NO: 4 (or SEQ ID NO: 64 or 66) fused to the N-terminus of the Fc domain and the amino acid sequence of SEQ ID NO: 2 (or SEQ ID NO: 63 or 65) fused to the C-terminus of the Fc domain.

One or more FGF21 domains (two or more, three or more, etc.) may be fused to N-terminus and/or the C-terminus of the Fc domain (optionally via a linker), and the FGF21 domains may comprise the amino acid sequence of SEQ ID NO: 2 or SEQ ID NO: 4 (or any other FGF21 domain sequence referenced herein). For example, the disclosure contemplates a complex wherein the first monomer and/or the second monomer comprises two or more FGF21 domains comprising SEQ ID NO: 2 and/or SEQ ID NO: 4 (e.g., a domain comprising SEQ ID NO: 2 fused with a domain comprising SEQ ID NO: 2 in tandem, a domain comprising SEQ ID NO: 4 fused with a domain comprising SEQ ID NO:4 in tandem, or a combination thereof) fused to the N-terminus of the Fc domain.

Alternatively, the first monomer comprises the amino acid sequence of SEQ ID NO: 4 (or SEQ ID NO: 64 or 66) fused to the N-terminus of the Fc domain and the amino acid sequence of SEQ ID NO: 4 (or SEQ ID NO: 64 or 66) fused to the C-terminus of the Fc domain. Optionally, (i) the second monomer comprises the amino acid sequence of SEQ ID NO: 4 (or SEQ ID NO: 64 or 66) fused to the N-terminus of the Fc domain or (ii) the second monomer comprises the amino acid sequence of SEQ ID NO: 2 (or SEQ ID NO: 63 or 65) fused to the C-terminus of the Fc domain. In various aspects, the second monomer comprises the amino acid sequence of SEQ ID NO: 4 (or SEQ ID NO: 64 or 66) fused to the N-terminus of the Fc domain and the amino acid sequence of SEQ ID NO: 4 (or SEQ ID NO: 64 or 66) fused to the C-terminus of the Fc domain.

In various aspects, the first monomer of the complex comprises the amino acid sequence of SEQ ID NO: 2 (or SEQ ID NO: 63 or 65) fused to the N-terminus of the Fc domain and the amino acid sequence of SEQ ID NO: 2 (or SEQ ID NO: 63 or 65) fused to the C-terminus of the Fc domain. Optionally, (i) the second monomer comprises the amino acid sequence of SEQ ID NO: 4 (or SEQ ID NO: 64 or 66) fused to the N-terminus of the Fc domain or (ii) the second monomer comprises the amino acid sequence of SEQ ID NO: 2 (or SEQ ID NO: 63 or 65) fused to the C-terminus of the Fc domain. In some embodiments, the second monomer comprises the amino acid sequence of SEQ ID NO: 2 (or SEQ ID NO: 63 or 65) fused to the N-terminus of the Fc domain and the amino acid sequence of SEQ ID NO: 2 (or SEQ ID NO: 63 or 65) fused to the C-terminus of the Fc domain.

The disclosure provides a complex comprising a two monomers comprising the amino acid sequence of SEQ ID NO: 18, a complex comprising two monomers comprising the amino acid sequence of SEQ ID NO: 19, and a complex comprising two monomers comprising the amino acid sequence of SEQ ID NO: 20. In various aspects of the disclosure, the monomers comprise an amino acid sequence at least about 90% identical (e.g., at least about 91%, at least about 92%, at least about 93%, at least about 94%, at least about 95%, at least about 96%, at least about 97%, at least about 98%, at least about 99% or 100%) identical to SEQ ID NO: 18, 19, or 20. For example, the disclosure contemplates monomers wherein one or more of the FGF21 domain(s) is the L-isoform of the FGF domain(s) within SEQ ID NOs: 18-20.

The FGF21 domain is fused to an Fc domain. An “Fc region” or “Fc domain” is a polypeptide comprising the constant region of an antibody, e.g., at least the second and third constant region domains of an antibody (CH2 and CH3 regions), and all or part of the hinge. Thus, an Fc domain can refer to the last two antibody constant region domains (e.g., CH2 and CH3) of IgA, IgD, and IgG, the last three antibody constant region domains of IgE and IgM, and the hinge N-terminal to these domains, in various aspects of the disclosure. For IgA and IgM, the Fc domain may include the J chain. For IgG, the Fc domain may comprise immunoglobulin domains Cy2 and Cy3 and the lower hinge region between Cγ1 and Cγ2. In some aspects of the disclosure, the Fc domain comprises the CH2 and CH3 domains with a truncated CH1 domain. Merely to illustrate, the human IgG Fc region is usually defined to include residues E216 or C226 or P230 and extend to the C-terminus (numbering is according to the EU index as in Kabat); CH1 generally corresponds to positions 118-220 according to the EU index as in Kabat, CH2 generally corresponds to positions 237-340 according to the EU index as in Kabat, and CH3 generally corresponds to positions 341-447 according to the EU index as in Kabat, all in the context of IgG. The Fc domain is preferably derived from an IgG Fc domain, e.g., IgG1, IgG2, IgG3 or IgG4 Fc domain. In an exemplary aspect of the disclosure, the Fc domain is an IgG1 Fc domain. A representative Fc domain of the disclosure is provided as SEQ ID NO: 7. The Fc domain optionally comprises an amino acid sequence at least about 90% identical (e.g., at least about 91%, at least about 92%, at least about 93%, at least about 94%, at least about 95%, at least about 96%, at least about 97%, at least about 98%, at least about 99% or 100%) identical to SEQ ID NO: 7. In any of the aspects described herein, one or both of the Fc domains may comprise the amino acid sequence of SEQ ID NO: 7. The Fc domain may be a variant of SEQ ID NO: 7, comprising modifications which optionally further extend half-life of the molecule. The Fc domain of the disclosure alternatively may comprise the amino acid sequence of SEQ ID NOs: 76-83. The complex may be dimeric or multimeric. The disclosure contemplates a complex wherein the monomers of the disclosure associate to form an antibody-like dimeric Fc domain structure. To illustrate, the disclosure contemplates two FGF21 domains each fused to an Fc domain, and Fc domains of two monomers associate to form an antibody-like structure (or complex) wherein the FGF21 binding domains are fused to, e.g., each CH2-CH3 arm, providing four FGF21 domains available for binding to beta-klotho or FGFR1c.

In any of the aspects of the disclosure described herein, the FGF21 domain(s) and Fc domain are optionally fused via a linker. A “linker” is an amino acid sequence which links multiple peptide domains, such as an FGF21 domain and an Fc domain or two FGF21 domains. Any suitable linker can be used so long as the FGF21 domain retains biological activity (e.g., retains the ability to bind beta-klotho and/or FGFR1c). The linker may be any length, e.g., 1-60 amino acids, such as 1-5, 1-10, 2-18, 4-30, 5-15, or 10-25 amino acids, in length. Suitable linkers include, but are not limited to, glycine-serine polymers, including for example (GS)n, (GGGGS)n (also referenced as “G4S,” SEQ ID NO: 67), (SGGGG)n (SEQ ID NO: 68), (GSSSS)n (SEQ ID NO: 69), and (GGGS)n (also referenced as “G3S,” SEQ ID NO: 70), where n is an integer of at least one (e.g., one, two, three, four, five, six, seven, eight, nine, or ten). Examples of linkers include, but are not limited to, GGGGGGGGS (SEQ ID NO: 71), GGGGSGGGGSGGGGS (SEQ ID NO: 72), GGGGSGGGGSGGGGSGGGGS (SEQ ID NO: 73), and GGGGSGGGGSGGGGSGGGGSGGGGS (SEQ ID NO: 74). In any complex or peptide described herein, one or more FGF21 domains may be fused to an Fc domain via a linker comprising the amino acid sequence of SEQ ID NO: 9 or SEQ ID NO: 10 or both. While the description above references of use of linkers to adjoin FGF21 domains or FGF21 domains and Fc domains, linker sequences also may be appended to the N-terminus and/or C-terminus of the monomers.

Also provided herein is a peptide comprising an Fc domain (e.g., an Fc domain comprising the amino acid sequence of SEQ ID NO: 7 or SEQ ID NO: 76-83) fused to an FGF21 domain comprising the amino acid sequence of SEQ ID NO: 4 (or SEQ ID NO: 64 or 66) at the N-terminus and an FGF21 domain comprising the amino acid sequence of SEQ ID NO:2 at the C-terminus. In various aspects, the FGF21 domain comprising SEQ ID NO:4 is fused to the Fc domain via a linker, such as any of the linkers described herein (e.g., a linker comprising the amino acid sequence of SEQ ID NO: 10), and the FGF21 domain comprising SEQ ID NO: 2 (or SEQ ID NO: 63 or 65) is fused to the Fc domain via a linker, such as any of the linkers described herein (e.g., a linker comprising the amino acid sequence of SEQ ID NO: 9). For example, the disclosure provides a peptide comprising the amino acid sequence of SEQ ID NO: 21. In various aspects of the disclosure, the disclosure provides a peptide comprising an amino acid sequence at least about 90% identical (e.g., at least about 91%, at least about 92%, at least about 93%, at least about 94%, at least about 95%, at least about 96%, at least about 97%, at least about 98%, at least about 99% or 100%) identical to SEQ ID NO: 21. For example, the disclosure contemplates monomers wherein one or more of the FGF21 domain(s) is the L-isoform of the FGF domain(s) within SEQ ID NO: 21. The disclosure further provides a complex comprising the peptide and a second Fc domain.

The disclosure further provides a complex comprising (a) a first monomer comprising an Fc domain (e.g., an Fc domain comprising the amino acid sequence of SEQ ID NO: 7 or SEQ ID NO: 76-83) fused at the C-terminus with an FGF21 domain comprising the amino acid sequence of SEQ ID NO: 4 (or SEQ ID NO: 64 or 66) and (b) a second monomer comprising an Fc domain (e.g., an Fc domain comprising the amino acid sequence of SEQ ID NO: 7 or SEQ ID NO: 76-83) fused at the N-terminus with an FGF21 domain comprising the amino acid sequence of SEQ ID NO: 2 (or SEQ ID NO: 63 or 65). In this regard, the FGF21 domain comprising SEQ ID NO: 4 is optionally fused to the Fc domain of the first monomer via a linker, such as any of the linkers described herein (e.g., a linker comprising the amino acid sequence of SEQ ID NO: 10). The FGF21 domain comprising SEQ ID NO: 2 (or SEQ ID NO: 63 or 65) is optionally fused to the Fc domain of the second monomer via a linker, such as any of the linkers described herein (e.g., a linker comprising the amino acid sequence of SEQ ID NO: 9). The disclosure provides a complex the first monomer comprises the amino acid sequence of SEQ ID NO: 27 and the second monomer comprises the amino acid sequence of SEQ ID NO: 28. In various aspects of the disclosure, the disclosure provides monomers comprising an amino acid sequence at least about 90% identical (e.g., at least about 91%, at least about 92%, at least about 93%, at least about 94%, at least about 95%, at least about 96%, at least about 97%, at least about 98%, at least about 99% or 100%) identical to SEQ ID NO: 27 or 28. For example, the disclosure contemplates monomers wherein one or more of the FGF21 domain(s) is the L-isoform of the FGF domain(s) within SEQ ID NO: 21.

Also provided herein is a peptide comprising the amino acid sequence of SEQ ID NO: 4 (or SEQ ID NO: 64 or 66) fused to the amino acid sequence of SEQ ID NO: 2 (or SEQ ID NO: 63 or 65) via a linker, such as any one or more of the linkers described herein (e.g., a linker comprising the amino acid sequence of SEQ ID NO: 9 and/or a linker comprising the amino acid sequence of SEQ ID NO: 10).

The disclosure further provides nucleic acids (i.e., polynucleotides) which comprise a nucleic acid sequence that encodes any of the domains, monomers, and peptides described herein. The disclosure further provides an expression vector comprising a nucleic acid described herein. For instance, the disclosure provides a nucleic acid comprising a nucleotide sequence encoding the first monomer of any complex described above, as well as an expression vector comprising the nucleic acid. The disclosure further provides a nucleic acid comprising a nucleotide sequence encoding the second monomer of any of the complexes described herein, as well as an expression vector comprising the nucleic acid. Additionally, the disclosure provides a system or composition comprising an expression vector comprising a nucleic acid comprising a nucleotide sequence encoding the first monomer of any of the complexes described herein and an expression vector comprising a nucleic acid comprising a nucleotide sequence encoding the second monomer of any of the complexes described herein. In this regard, the disclosure provides a system comprising (a) an expression vector comprising a nucleic acid comprising a nucleic acid sequence encoding a first monomer comprising an Fc domain fused to two FGF21 domains comprising the amino acid sequence of SEQ ID NO: 2 (or SEQ ID NO: 63 or 65) or SEQ ID NO: 4 (or SEQ ID NO: 64 or 66), which may be the same or different (e.g., a first monomer comprises the amino acid sequence of SEQ ID NO: 4 fused to the N-terminus of the Fc domain and the amino acid sequence of SEQ ID NO: 2 (or SEQ ID NO: 63 or 65) fused to the C-terminus of the Fc domain; a first monomer comprises the amino acid sequence of SEQ ID NO: 4 (or SEQ ID NO: 64 or 66) fused to the N-terminus of the Fc domain and the amino acid sequence of SEQ ID NO: 4 (or SEQ ID NO: 64 or 66) fused to the C-terminus of the Fc domain; a first monomer comprises the amino acid sequence of SEQ ID NO: 2 (or SEQ ID NO: 63 or 65) fused to the N-terminus of the Fc domain and the amino acid sequence of SEQ ID NO: 2 (or SEQ ID NO: 63 or 65) fused to the C-terminus of the Fc domain; or a first monomer comprising an Fc domain fused at the C-terminus with an FGF21 domain comprising the amino acid sequence of SEQ ID NO: 4 (or SEQ ID NO: 64 or 66)), and (b) an expression vector comprising a nucleic acid comprising a nucleotide sequence encoding a second monomer comprising an Fc domain fused to at least one FGF21 domain comprising the amino acid sequence of SEQ ID NO: 2 (or SEQ ID NO: 63 or 65) or SEQ ID NO: 4 (or SEQ ID NO: 64 or 66) (e.g., fused to an FGF21 domain comprising the amino acid sequence of SEQ ID NO: 2 (or SEQ ID NO: 63 or 65) or SEQ ID NO: 4 (or SEQ ID NO: 64 or 66) at the N-terminus and/or fused to an FGF21 domain comprising the amino acid sequence of SEQ ID NO: 2 (or SEQ ID NO: 63 or 65) or SEQ ID NO: 4 (or SEQ ID NO: 64 or 66) at the C-terminus).

Expression vectors provide for expression in vitro and/or in vivo (e.g., in a suitable host cell or organism). Many expression vectors are commercially available. The nucleic acid molecule may be provided in e.g., a plasmid, cosmid, YAC, or a viral vector. Suitable viral vectors include, for example, retrovirus, adenovirus, parvovirus (for example, adeno-associated viruses), coronavirus, negative strand RNA viruses such as ortho-mycovirus (for example, influenza virus), rhabdovirus (for example, rabies and vesicular stomatitis virus), paramyxovirus (for example, measles and Sendai), picornavirus, alphavirus, herpesvirus (for example, Herpes Simplex virus types 1 and 2, Epstein-Barr virus, cytomegalovirus), lentivirus, and poxvirus (for example, vaccinia, fowlpox, and canarypox). Vector components may include an origin of replication, one or more marker genes, a multiple cloning site containing recognition sequences for restriction endonucleases, enhancer elements, promoters, transcription termination sequences, and the like. Indeed, the nucleic acid of the disclosure may be operably linked to one or more regulatory elements, such as a promoter, enhancer, and/or terminator.

The disclosure further provides a host cell comprising the nucleic acid(s) or the expression vector(s) described herein. A “host cell” refers to a cell (e.g., prokaryotic or eukaryotic) into which exogenous nucleic acid has been introduced, including the progeny of such cells. A host cell may be a bacterial cell, a yeast cell, an insect cell, or a mammalian cell. In various aspects, the cell is a eukaryotic cell, such as a mammalian cell (e.g., a human cell; or cell from a non-human primate such as ape, chimpanzee, monkey, or orangutan; or cell from a domesticated animal, such as a dog or cat; or cell from livestock, such as a horse, cow, pig, sheep, or goat; or cell from another mammalian species including, without limitation, mice, rats, guinea pigs, rabbits, hamsters, birds (e.g., chicken, duck, goose, quail or pheasant) and the like). A representative bacterial host cell is E. coli. Examples of human cells include, but are not limited to, PER.C6 cells (described in, e.g., International Patent Publication No. WO 01/38362), MRC-5 (ATCC CCL-171), WI-38 (ATCC CCL-75), HEK-293 cells (ATCC CRL-1573), Hela cells (ATCC CCL2), and fetal rhesus lung cells (ATCC CL-160). Examples of non-human primate cells are Vero cells (ATCC CCL81), COS-1 cells (ATCC CRL-1650), and COS-7 cells (ATCC CRL-1651). Examples of dog cells are MDCK cells (ATCC CCL-34). Examples of rodent cells are hamster cells, such as BHK21-F, HKCC cells, or Chinese hamster ovary (CHO) cells. Examples of insect cells include, but are not limited to, SF9 cells (ATCC CRL-1711), Sf21 cells (IPLB-Sf21), MG1 cells (BTI-TN-MG1), and High Five™ cells (BTI-TN-5B1-4).

Also contemplated is a method of producing a FGF21 domain-comprising complex described herein, the method comprising culturing a host cell comprising the expression vector(s) or system described herein and harvesting the complex. Similarly, the disclosure provides a method of producing a FGF21 domain-comprising peptide described herein, the method comprising culturing a host cell comprising the expression vector(s) or system described herein and harvesting the peptide. Culture conditions and methods for generating recombinant proteins are known in the art. Similarly, protein purification methods are known in the art and utilized herein for recovery of recombinant proteins from cell culture media. In some aspects, methods for protein purification include filtration, affinity column chromatography, cation exchange chromatography, anion exchange chromatography, and concentration. Optionally, the method comprises formulating the recovered product.

The disclosure further provides a pharmaceutical composition comprising the complex or peptide described herein. Such pharmaceutical compositions can comprise a therapeutically effective amount of the complex or peptide described herein in admixture with a pharmaceutically or physiologically acceptable formulation agent selected for suitability with a mode of administration. Also provided is a pharmaceutical composition comprising a nucleic acid (e.g., mRNA) encoding any one or more of the monomers or peptides described herein.

The pharmaceutical composition optionally comprises components for modifying, maintaining, or preserving, for example, the pH, osmolarity, viscosity, clarity, color, isotonicity, odor, sterility, stability, rate of dissolution or release, adsorption, or penetration of the composition. Suitable formulation components include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, trehalose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thiomersal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, including sodium or potassium chloride or mannitol sorbitol), delivery vehicles, diluents, excipients and/or pharmaceutical adjuvants (see, e.g., Remington's Pharmaceutical Sciences (18th Ed., A. R. Gennaro, ed., Mack Publishing Company 1990), and subsequent editions of the same, incorporated herein by reference for any purpose).

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer (e.g., of about pH 7.0-8.5), or acetate buffer (e.g., of about pH 4.0-5.5). In various aspects of the disclosure, the FGF21 domain-comprising complex or peptide may be prepared for storage by mixing the selected composition having the desired degree of purity with optional formulation agents (Remington's Pharmaceutical Sciences, supra) in the form of a lyophilized cake or an aqueous solution. Further, the FGF21 polypeptide mutant product may be formulated as a lyophilizate.

The complex or peptide disclosed herein can be used to treat, ameliorate, prevent or reverse a number of diseases, disorders, or conditions, including, but not limited to genetic disorders, metabolic disorders, and fibrotic disorders. In various aspects, the disclosure provides a method of treating a disease or disorder, wherein the method comprises administering a pharmaceutical composition comprising the complex or peptide described herein to a subject (e.g., a human) in need thereof. The disease or disorder may be any following: non-alcoholic steatohepatitis (NASH), nonalcoholic fatty liver disease (NAFLD), metabolic dysfunction-associated steatohepatitis (MASH), metabolic dysfunction-associated steatotic liver disease (MASLD), metabolic-dysfunction associated alcoholic liver disease (MetALD), cryptogenic steatotic liver disease (SLD), hepatic steatosis, alcoholic steatohepatitis (ASH), alcoholic liver disease (ALD) or alcoholic fatty liver disease (AFLD), diabetes (e.g., type 2 diabetes), obesity, hypertriglyceridemia, dyslipidemia, cardiovascular disease (such as atherosclerosis), or aging. In various aspects, the disclosure provides a method of reversing liver cirrhosis or reducing fibrosis, such as liver fibrosis associated with NASH, NAFLD, MASH, MASLD, MetALD, ASH, ALD, or AFLD, as well as liver fibrosis associated with non-metabolic disorders. Optionally, following treatment, the subject's fibrosis score, based on NASH Clinical Research Network (CRN) histological scoring system, (Kleiner D et al, 2005 Hepatology 41, 1313), may regress from F4 (cirrhosis) to F3 (advanced fibrosis) or less. Fibrosis of other organs or tissues (e.g., lung, pancreas, or heart) is also contemplated. The disclosure also contemplates a method of alleviating or minimizing cravings or addictions (alcohol-related or other, such as food) by administering a pharmaceutical composition comprising the complex or peptide described herein. In exemplary embodiments, addiction encompasses persistent, compulsive dependence on a behavior or substance such as alcohol, drugs, or nicotine. In exemplary embodiments, craving encompasses a strong, urgent, or abnormal desire for a certain substance or activity, such as sugar.

The disclosure also provides a method of normalizing liver fat content, reducing blood glucose levels, increasing insulin sensitivity, and/or reducing uric acid levels, by administering a pharmaceutical composition comprising the complex or peptide disclosed herein to a subject in need thereof. In exemplary embodiments, normalizing liver fat content refers to reducing liver fat content (e.g., absolute liver fat content), preferably reducing liver fat content to that of a typical, healthy, non-diseased subject (i.e. a subject not suffering from one or more of the diseases/disorders described herein). In various embodiments, liver fat content is reduced to <5% absolute liver fat. An absolute liver fat content of >5% is associated with hepatic steatosis (fatty liver disease), with <5% absolute liver fat content being considered within a clinically normal range for non-diseased subjects (see, for example, Chalasani et al., 2018 Hepatology; 67(1):328-357).

A “subject in need thereof” is a subject, such as a human, that would benefit from the administration of the pharmaceutical composition, and may be diagnosed with or suffering from symptoms of any of the disorders described herein. For example, the subject in need of reducing uric acid levels may be a subject suffering from gout. The subject in need of a method of reversing liver cirrhosis or reducing fibrosis associated with NASH, MASH, ASH, ALD, or AFLD may be suffering from NASH, MASH, ASH, ALD, or AFLD, or recovering from NASH, ASH, ALD, or ALFD.

The term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease or disorder can provide any amount or any level of treatment. Furthermore, the treatment provided by the method may include treatment of one or more conditions or symptoms or signs of the disease being treated and/or improving quality of life of the subject with the condition or disease. The treatment method of the present disclosure may inhibit one or more symptoms of the disease. Also, the treatment provided by the methods of the present disclosure may encompass slowing or reversing progression of the disease.

Improvement of quality of life of a subject can be measured by determining one or more quality of life parameters using, for instance, the European Quality of Life 5 questions tool (EQ-5D) to determine mobility, mood, holistic impact on patients' quality of life as reported by patients. The EQ-5D questionnaire also includes a Visual Analog Scale (VAS), by which respondents can report their perceived health status. See, for example, Balestroni et al., Monaldi Arch Chest Dis. 2012 September; 78(3):155-9, which is incorporated by reference in its entirety. Treatment may also be monitored using a Liver Disease Questionnaire. See, for example, Younossi et al., Clin Gastroenterol Hepatol. 2019 September; 17(10):2093-2100.e3, which is incorporated by reference in its entirety. Liver treatment also may be monitored by measuring objective parameters such as histology data (e.g., regression of fibrosis, resolution of NASH, and the like) and non-invasive biomarkers of liver fibrosis (Pro-C3, ELF), injury (e.g., alanine aminotransferase (ALT), aspartate transaminase (AST), gamma-glutamyl transferase (GGT), and/or alkaline phosphatase (ALP)), stiffness (e.g., VCTE, Fibroscan), and inflammation and fibrosis (e.g., corrected T1 by MRI). Exemplary methods of histopathology scoring of liver in NASH patients are disclosed in, for example, Kleiner et al, 2005 Hepatology 41, 1313, which is incorporated by reference in its entirety.

With regard to the foregoing methods, the composition comprising the complex or peptide may be administered by any suitable route of administration, including intravenous, intraperitoneal, intracerebral (intra-parenchymal), intramuscular, intra-ocular, intraarterial, intraportal, intramedullary, intrathecal, intraventricular, intradermal, transdermal, subcutaneous, intranasal, inhalation (e.g., upper and/or lower airways), enteral, epidural, urethral, vaginal, or rectal routes of administration. In various instances, the composition is administered to the subject intravenously, intramuscularly, or subcutaneously. For example, in some aspects, the composition is administered subcutaneously. The amount or dose of complex or peptide in the composition (i.e., the “effective amount”) administered should be sufficient to achieve a desired biological effect in the subject over a clinically reasonable time frame (e.g., about 0.1 mg to about 100 mg).

In jurisdictions that forbid the patenting of methods that are practiced on the human body, the meaning of “administering” a composition to a human subject may be restricted to prescribing a controlled substance that a human subject can self-administer by any technique (e.g., injection, insertion, etc.). The disclosure contemplates use of the pharmaceutical composition to treat any of the diseases or disorders described here. The disclosure further contemplates use of the composition in the preparation of a medicament for treating any of the diseases or disorders described herein. The disclosure further provides the composition described herein for use in the treatment of any of the diseases or disorders referenced here. In jurisdictions that do not forbid the patenting of methods that are practiced on the human body, the “administering” of compositions includes both methods practiced on the human body and also the foregoing activities.

As an additional aspect, kits are provided which comprise a pharmaceutical composition comprising the complex or peptide described herein packaged in a manner which facilitates administration to subjects. In one aspect, the kit includes a pharmaceutical composition/formulation comprising the complex or peptide described herein packaged in a container such as a sealed bottle, vessel, single-use or multi-use vial, prefilled device (e.g. a single or dual chamber syringe or dual chamber cartridge for use in an automated injection device), or prefilled injection device, optionally with a label affixed to the container or included in the package that describes use of the pharmaceutical composition in practicing the method. In one aspect, the composition comprising the complex or peptide described herein is packaged in a unit dosage form. The kit may include a device suitable for administering a pharmaceutical composition according to a specific route of administration, although this is not required.

Various sequences referenced in the application are provided in Table 1.

TABLE 1

| SEQ ID | Sequence |
|---|---|
| 1 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSHPIPDSSPLLQF |
| | GGQVRQRYLYTDDAQQTEAHLEIREDGTVGGAADQSPESLLQLKALKPGVIQILGVKTSRFLCQ |
| | RPDGALYGSLHFDPEACSFRERLLEDGYNVYQSEAHGLPLHLPGNKSPHRDPAPRGPARFLPLP |
| | GLPPAPPEPPGILAPQPPDVGSSDPLSMVGGSQGRSPSYES |
| 2 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYES |
| 3 | QGRSPSYES |
| 4 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPH |
| 5 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPH |
| 6 | SEYSPSRGQ |
| 7 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 8 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 9 | GGGGSGGGGSGGGGS |
| 10 | SGGGGSGGGGSGGGG |
| 11 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSHPIPDSSPLLQFG |
| | GQVRQRYLYTDDAQQTEAHLEIREDGTVGGAADQSPESLLQLKALKPGVIQILGVKTSRFLCQRP |
| | DGALYGSLHFDPEACSFRERLLEDGYNVYQSEAHGLPLHLPGNKSPHRDPAPRGPARFLPLPGLP |
| | PAPPEPPGILAPQPPDVGSSDPLSMVGGSQGRSPSYES |
| 12 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 13 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 14 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSHPIPDSSPLLQFG |
| | GQVRQRYLYTDDAQQTEAHLEIREDGTVGGAADQSPESLLQLKALKPGVIQILGVKTSRFLCQRP |
| | DGALYGSLHFDPEACSFRERLLEDGYNVYQSEAHGLPLHLPGNKSPHRDPAPRGPARFLPLPGLP |
| | PAPPEPPGILAPQPPDVGSSDPLSMVGGSQGRSPSYES |
| 15 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSSEYSPSRGQSGGV |
| | MSLPDSSGVDPPQPALIGPPEPPAPPLGPLPLFRAPGRPAPDRHPSKNGPLHLPLGHAESQYVN |
| | YGDELLRERFSCAEPDFHLSGYLAGDPRQCLFRSTKVGLIQIVGPKLAKLQLLSEPSQDAAGGVTG |
| | DERIELHAETQQADDTYLYRQRVQGGFQLLPSSDPIPH |
| 16 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 17 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 18 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGKGGGGSGGGGGGGGSHPIPDSSPLL |
| | QFGGQVRQRYLYTDDAQQTEAHLEIREDGTVGGAADQSPESLLQLKALKPGVIQILGVKTSRFL |
| | CORPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 19 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSGGGGSHPIPDSSPL |
| | LQFGGQVRQRYLYTDDAQQTEAHLEIREDGTVGGAADQSPESLLQLKALKPGVIQILGVKTSRFL |
| | CQRPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 20 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGKGGGGSGGGGSGGGGSSEYSPSRGQ |
| | SGGVMSLPDSSGVDPPQPALIGPPEPPAPPLGPLPLFRAPGRPAPDRHPSKNGPLHLPLGHAES |
| | QYVNYGDELLRERFSCAEPDFHLSGYLAGDPRQCLFRSTKVGLIQIVGPKLAKLQLLSEPSQDAA |
| | GGVTGDERIELHAETQQADDTYLYRQRVQGGFQLLPSSDPIPH |
| 21 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGKGGGGSGGGGSGGGGSHPIPDSSPLL |
| | QFGGQVRQRYLYTDDAQQTEAHLEIREDGTVGGAADQSPESLLQLKALKPGVIQILGVKTSRFL |
| | CQRPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 22 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 23 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSGGGGSHPIPDSSPL |
| | LQFGGQVRQRYLYTDDAQQTEAHLEIREDGTVGGAADQSPESLLQLKALKPGVIQILGVKTSRFL |
| | CORPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 24 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 25 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGKGGGGGGGGSGGGGSSEYSPSRGQ |
| | SGGVMSLPDSSGVDPPQPALIGPPEPPAPPLGPLPLFRAPGRPAPDRHPSKNGPLHLPLGHAES |
| | QYVNYGDELLRERFSCAEPDFHLSGYLAGDPRQCLFRSTKVGLIQIVGPKLAKLQLLSEPSQDAA |
| | GGVTGDERIELHAETQQADDTYLYRQRVQGGFQLLPSSDPIPH |
| 26 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| 27 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 28 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSHPIPDSSPLLQFG |
| | GQVRQRYLYTDDAQQTEAHLEIREDGTVGGAADQSPESLLQLKALKPGVIQILGVKTSRFLCQRP |
| | DGALYGSLHFDPEACSFRERLLEDGYNVYQSEAHGLPLHLPGNKSPHRDPAPRGPARFLPLPGLP |
| | PAPPEPPGILAPQPPDVGSSDPLSMVGGSQGRSPSYES |
| 29 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 30 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSHPIPDSSPLLQFG |
| | GQVRQRYLYTDDAQQTEAHLEIREDGTVGGAADQSPESLLQLKALKPGVIQILGVKTSRFLCQRP |
| | DGALYGSLHFDPEACSFRERLLEDGYNVYQSEAHGLPLHLPGNKSPHRDPAPRGPARFLPLPGLP |
| | PAPPEPPGILAPQPPDVGSSDPLSMVGGSQGRSPSYES |
| 31 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGDKTHTCPPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDGSFFLYSKL |
| | TVDKSRWQQGNVFSCSVMHEALHNHYTQKSLSLSPGK |
| 32 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSSEYSPSRGQSGGV |
| | MSLPDSSGVDPPQPALIGPPEPPAPPLGPLPLFRAPGRPAPDRHPSKNGPLHLPLGHAESQYVN |
| | YGDELLRERFSCAEPDFHLSGYLAGDPRQCLFRSTKVGLIQIVGPKLAKLQLLSEPSQDAAGGVTG |
| | DERIELHAETQQADDTYLYRQRVQGGFQLLPSSDPIPH |
| 33 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 34 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVMHEALHNHYTQKSLSLSPGKGGGGSGGGGSGGGGSSEYSPSRGQSGGV |
| | MSLPDSSGVDPPQPALIGPPEPPAPPLGPLPLFRAPGRPAPDRHPSKNGPLHLPLGHAESQYVN |
| | YGDELLRERFSCAEPDFHLSGYLAGDPRQCLFRSTKVGLIQIVGPKLAKLQLLSEPSQDAAGGVTG |
| | DERIELHAETQQADDTYLYRQRVQGGFQLLPSSDPIPH |
| 35 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGSGGGGSG |
| | GGGGGGGSGGGGSGGGGSHPIPDSSPLLQFGGQVRQRYLYTDDAQQTEAHLEIREDGTVGGA |
| | ADQSPESLLQLKALKPGVIQILGVKTSRFLCQRPDGALYGSLHFDPEACSFRERLLEDGYNVYQSE |
| | AHGLPLHLPGNKSPHRDPAPRGPARFLPLPGLPPAPPEPPGILAPQPPDVGSSDPLSMVGGSQG |
| | RSPSYES |
| 36 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSHPIPDSSPLLQF |
| | GGQVRQRYLYTDDAQQTEAHLEIREDGTVGGAADQSPESLLQLKALKPGVIQILGVKTSRFLCQ |
| | RPDGALYGSLHFDPEACSFRERLLEDGYNVYQSEAHGLPLHLPGNKSPHRDPAPRGPARFLPLP |
| | GLPPAPPEPPGILAPQPPDVGSSDPLSMVGGSQGRSPSYES |
| 37 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGS |
| 38 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 39 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSHPIPDSSPLLQF |
| | GGQVRQRYLYTDDAQQTEAHLEIREDGTVGGAADQSPESLLQLKALKPGVIQILGVKTSRFLCQ |
| | RPDGALYGSLHFDPEACSFRERLLEDGYNVYQSEAHGLPLHLPGNKSPHRDPAPRGPARFLPLP |
| | GLPPAPPEPPGILAPQPPDVGSSDPLSMVGGSQGRSPSYES |
| 40 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSSEYSPSRGQSG |
| | GVMSLPDSSGVDPPQPALIGPPEPPAPPLGPLPLFRAPGRPAPDRHPSKNGPLHLPLGHAESQY |
| | VNYGDELLRERFSCAEPDFHLSGYLAGDPRQCLFRSTKVGLIQIVGPKLAKLQLLSEPSQDAAGG |
| | VTGDERIELHAETQQADDTYLYRQRVQGGFQLLPSSDPIPH |
| 41 | MHPIPDSSPLLQFGGQVRQRYLYTDDAQQTEAHLEIREDGTVGGAADQSPESLLQLKALKPGVI |
| | QILGVKTSRFLCQRPDGALYGSLHFDPEACSFRERLLEDGYNVYQSEAHGLPLHLPGNKSPHRDP |
| | APRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 42 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 43 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGKGGGGSGGGGSGGGGSHPIPDSSP |
| | LLQFGGQVRQRYLYTDDAQQTEAHLEIREDGTVGGAADQSPESLLQLKALKPGVIQILGVKTSRF |
| | LCQRPDGALYGSLHFDPEACSFRERLLEDGYNVYQSEAHGLPLHLPGNKSPHRDPAPRGPARFL |
| | PLPGLPPAPPEPPGILAPQPPDVGSSDPLSMVGGSQGRSPSYES |
| 44 | MHPIPDSSPLLQFGGQVRQRYLYTDDAQQTEAHLEIREDGTVGGAADQSPESLLQLKALKPGVI |
| | QILGVKTSRFLCQRPDGALYGSLHFDPEACSFRERLLEDGYNVYQSEAHGLPLHLPGNKSPHRDP |
| | APRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSGGGGSHPIPDSSPL |
| | LQFGGQVRQRYLYTDDAQQTEAHLEIREDGTVGGAADQSPESLLQLKALKPGVIQILGVKTSRFL |
| | CORPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 45 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGKGGGGSGGGGSGGGGSSEYSPSRG |
| | QSGGVMSLPDSSGVDPPQPALIGPPEPPAPPLGPLPLFRAPGRPAPDRHPSKNGPLHLPLGHAE |
| | SQYVNYGDELLRERFSCAEPDFHLSGYLAGDPRQCLFRSTKVGLIQIVGPKLAKLQLLSEPSQDAA |
| | GGVTGDERIELHAETQQADDTYLYRQRVQGGFQLLPSSDPIPH |
| 46 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGKGGGGSGGGGSGGGGSHPIPDSSP |
| | LLQFGGQVRQRYLYTDDAQQTEAHLEIREDGTVGGAADQSPESLLQLKALKPGVIQILGVKTSRF |
| | LCQRPDGALYGSLHFDPEACSFRERLLEDGYNVYQSEAHGLPLHLPGNKSPHRDPAPRGPARFL |
| | PLPGLPPAPPEPPGILAPQPPDVGSSDPLSMVGGSQGRSPSYES |
| 47 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 48 | MHPIPDSSPLLQFGGQVRQRYLYTDDAQQTEAHLEIREDGTVGGAADQSPESLLQLKALKPGVI |
| | QILGVKTSRFLCQRPDGALYGSLHFDPEACSFRERLLEDGYNVYQSEAHGLPLHLPGNKSPHRDP |
| | APRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGKGGGGSGGGGSGGGGSHPIPDSSPL |
| | LQFGGQVRQRYLYTDDAQQTEAHLEIREDGTVGGAADQSPESLLQLKALKPGVIQILGVKTSRFL |
| | CQRPDGALYGSLHFDPEACSFRERLLEDGYNVYQSEAHGLPLHLPGNKSPHRDPAPRGPARFLP |
| | LPGLPPAPPEPPGILAPQPPDVGSSDPLSMVGGSQGRSPSYES |
| 49 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 50 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGKGGGGSGGGGSGGGGSSEYSPSRG |
| | QSGGVMSLPDSSGVDPPQPALIGPPEPPAPPLGPLPLFRAPGRPAPDRHPSKNGPLHLPLGHAE |
| | SQYVNYGDELLRERFSCAEPDFHLSGYLAGDPRQCLFRSTKVGLIQIVGPKLAKLQLLSEPSQDAA |
| | GGVTGDERIELHAETQQADDTYLYRQRVQGGFQLLPSSDPIPH |
| 51 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGS |
| 52 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 53 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSHPIPDSSPLLQF |
| | GGQVRQRYLYTDDAQQTEAHLEIREDGTVGGAADQSPESLLQLKALKPGVIQILGVKTSRFLCQ |
| | RPDGALYGSLHFDPEACSFRERLLEDGYNVYQSEAHGLPLHLPGNKSPHRDPAPRGPARFLPLP |
| | GLPPAPPEPPGILAPQPPDVGSSDPLSMVGGSQGRSPSYES |
| 54 | MHPIPDSSPLLQFGGQVRQRYLYTDDAQQTEAHLEIREDGTVGGAADQSPESLLQLKALKPGVI |
| | QILGVKTSRFLCQRPDGALYGSLHFDPEACSFRERLLEDGYNVYQSEAHGLPLHLPGNKSPHRDP |
| | APRGPARFLPLPGLPPAPPEPPGILAPQPPDVGSSDPLSMVGGSQGRSPSYESSGGGGGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 55 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSHPIPDSSPLLQF |
| | GGQVRQRYLYTDDAQQTEAHLEIREDGTVGGAADQSPESLLQLKALKPGVIQILGVKTSRFLCQ |
| | RPDGALYGSLHFDPEACSFRERLLEDGYNVYQSEAHGLPLHLPGNKSPHRDPAPRGPARFLPLP |
| | GLPPAPPEPPGILAPQPPDVGSSDPLSMVGGSQGRSPSYES |
| 56 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGDKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| 57 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSSEYSPSRGQSG |
| | GVMSLPDSSGVDPPQPALIGPPEPPAPPLGPLPLFRAPGRPAPDRHPSKNGPLHLPLGHAESQY |
| | VNYGDELLRERFSCAEPDFHLSGYLAGDPRQCLFRSTKVGLIQIVGPKLAKLQLLSEPSQDAAGG |
| | VTGDERIELHAETQQADDTYLYRQRVQGGFQLLPSSDPIPH |
| 58 | MHPIPDSSPLLQFGGQVRQRYLYTDDAQQTEAHLEIREDGTVGGAADQSPESLLQLKALKPGVI |
| | QILGVKTSRFLCQRPDGALYGSLHFDPEACSFRERLLEDGYNVYQSEAHGLPLHLPGNKSPHRDP |
| | APRGPARFLPLPGLPPAPPEPPGILAPQPPDVGSSDPLSMVGGSQGRSPSYESSGGGGSGGGGS |
| | GGGGDKTHTCPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 59 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGKGGGGSGGGGSGGGGSSEYSPSRGQSG |
| | GVMSLPDSSGVDPPQPALIGPPEPPAPPLGPLPLFRAPGRPAPDRHPSKNGPLHLPLGHAESQY |
| | VNYGDELLRERFSCAEPDFHLSGYLAGDPRQCLFRSTKVGLIQIVGPKLAKLQLLSEPSQDAAGG |
| | VTGDERIELHAETQQADDTYLYRQRVQGGFQLLPSSDPIPH |
| 60 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGGGGG |
| | SGGGGGGGGSGGGGSGGGGSHPIPDSSPLLQFGGQVRQRYLYTDDAQQTEAHLEIREDGTVG |
| | GAADQSPESLLQLKALKPGVIQILGVKTSRFLCQRPDGALYGSLHFDPEACSFRERLLEDGYNVY |
| | QSEAHGLPLHLPGNKSPHRDPAPRGPARFLPLPGLPPAPPEPPGILAPQPPDVGSSDPLSMVGG |
| | SQGRSPSYES |
| 61 | SEYSPSRGQSGGVMSLPDSSGVDPPQPALIGPPEPPAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPHSGGGGGGGGSG |
| | GGGXGGGGSGGGGSGGGGSHPIPDSSPLLQFGGQVRQRYLYTDDAQQTEAHLEIREDGTVGG |
| | AADQSPESLLQLKALKPGVIQILGVKTSRFLCQRPDGALYGSLHFDPEACSFRERLLEDGYNVYQS |
| | EAHGLPLHLPGNKSPHRDPAPRGPARFLPLPGLPPAPPEPPGILAPQPPDVGSSDPLSMVGGSQ |
| | GRSPSYES |
| 62 | MSEYSPSRGQSGGVMSLPDSSGVDPPQPALIGPPEPPAPPLGPLPLFRAPGRPAPDRHPSKNG |
| | PLHLPLGHAESQYVNYGDELLRERFSCAEPDFHLSGYLAGDPRQCLFRSTKVGLIQIVGPKLAKLQ |
| | LLSEPSQDAAGGVTGDERIELHAETQQADDTYLYRQRVQGGFQLLPSSDPIPHSGGGGSGGGG |
| | SGGGGXGGGGSGGGGSGGGGSHPIPDSSPLLQFGGQVRQRYLYTDDAQQTEAHLEIREDGTV |
| | GGAADQSPESLLQLKALKPGVIQILGVKTSRFLCQRPDGALYGSLHFDPEACSFRERLLEDGYNVY |
| | QSEAHGLPLHLPGNKSPHRDPAPRGPARFLPLPGLPPAPPEPPGILAPQPPDVGSSDPLSMVGG |
| | SQGRSPSYES |
| 63 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPA[X]PEPPGILAPQPPDVGSSDPLSMVGGSQGRSPSYES |
| | *[X] is P or L |
| 64 | SEYSPSRGQSGGVMSLPDSSGVDPPQPALIGPPEP[X]APPLGPLPLFRAPGRPAPDRHPSKNGP |
| | LHLPLGHAESQYVNYGDELLRERFSCAEPDFHLSGYLAGDPRQCLFRSTKVGLIQIVGPKLAKLQL |
| | LSEPSQDAAGGVTGDERIELHAETQQADDTYLYRQRVQGGFQLLPSSDPIPH |
| | *[X] is P or L |
| 65 | HPIPDSSPLLQFGGQVRQRYLYTDDAQQTEAHLEIREDGTVGGAADQSPESLLQLKALKPGVIQI |
| | LGVKTSRFLCQRPDGALYGSLHFDPEACSFRERLLEDGYNVYQSEAHGLPLHLPGNKSPHRDPA |
| | PRGPARFLPLPGLPPALPEPPGILAPQPPDVGSSDPLSMVGGSQGRSPSYES |
| 66 | SEYSPSRGQSGGVMSLPDSSGVDPPQPALIGPPEPLAPPLGPLPLFRAPGRPAPDRHPSKNGPL |
| | HLPLGHAESQYVNYGDELLRERFSCAEPDFHLSGYLAGDPRQCLFRSTKVGLIQIVGPKLAKLQLL |
| | SEPSQDAAGGVTGDERIELHAETQQADDTYLYRQRVQGGFQLLPSSDPIPH |
| 67 | GGGGS |
| 68 | SGGGG |
| 69 | GSSSS |
| 70 | GGGS |
| 71 | GGGGSGGGGS |
| 72 | GGGGSGGGGSGGGGS |
| 73 | GGGGSGGGGGGGGSGGGGS |
| 74 | GGGGSGGGGSGGGGSGGGGSGGGGS |
| 75 | [HPIPDSSPLLQFGGQVRQRYLYTDDAQQTEAHLEIREDGTVGGAADQSPESLLQLKALKPGVIQ |
| | ILGVKTSRFLCQRPDGALYGSLHFDPEACSFRERLLEDGYNVYQSEAHGLPLHLPGNKSPHRDPA |
| | PRGPARFLPLPGLPPAPPEPPGILAPQPPDVGSSDPLSMVGGSQGRSPSYESSGGGGSGGGGS |
| | GGGG]DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGKGGGGSGGGGSGGGGSHPIPDSSP |
| | LLQFGGQVRQRYLYTDDAQQTEAHLEIREDGTVGGAADQSPESLLQLKALKPGVIQILGVKTSRF |
| | LCQRPDGALYGSLHFDPEACSFRERLLEDGYNVYQSEAHGLPLHLPGNKSPHRDPAPRGPARFL |
| | PLPGLPPAPPEPPGILAPQPPDVGSSDPLSMVGGSQGRSPSYES - |
| | wherein multiple copies of bracketed sequence may |
| | be present in tandem |
| 76 | DKTHTCPPCPAPELLGGPSVFLFPPKPKDTLYITREPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 77 | DKTHTCPPCPAPELLGGPSVFLFPPKPKDQLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVLHEALHNHYTQKSLSLSPGK |
| 78 | DKTHTCPPCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFFLYSKLTVDK |
| | SRWQQGNVFSCSVLHEALHSHYTQKSLSLSPGK |
| 79 | DKTHTCPPCPAPELLGGPSVFLFPPKPKDTLYITREPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSKLTVDKS |
| | RWQQGNVFSCSVLHEALHSHYTQKSLSLSPGK |
| 80 | MDKTHTCPPCPAPELLGGPSVFLFPPKPKDTLYITREPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 81 | MDKTHTCPPCPAPELLGGPSVFLFPPKPKDQLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFLYSKLT |
| | VDKSRWQQGNVFSCSVLHEALHNHYTQKSLSLSPGK |
| 82 | MDKTHTCPPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVLHEALHSHYTQKSLSLSPGK |
| 83 | MDKTHTCPPCPAPELLGGPSVFLFPPKPKDTLYITREPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPVLDSDGSFFLYSKLTV |
| | DKSRWQQGNVFSCSVLHEALHSHYTQKSLSLSPGK |

### EXAMPLE

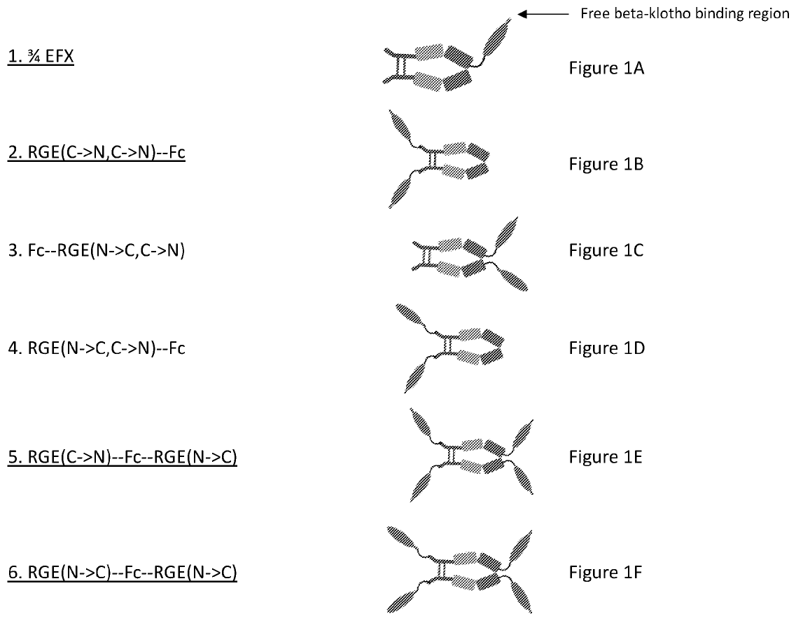

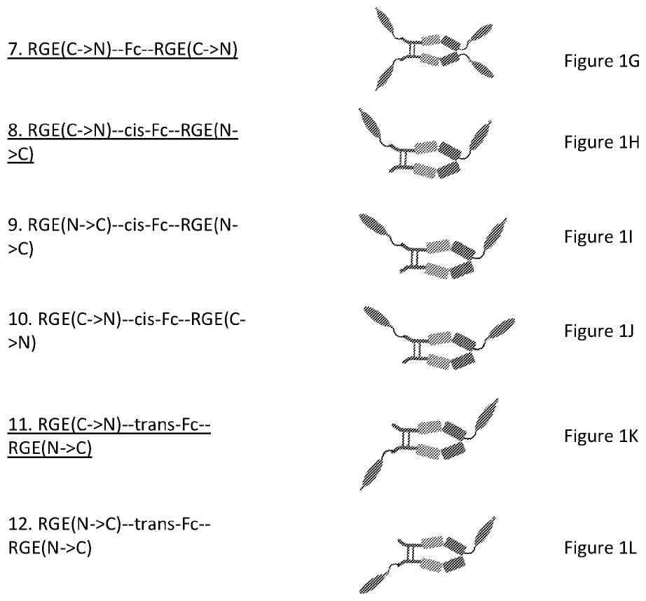

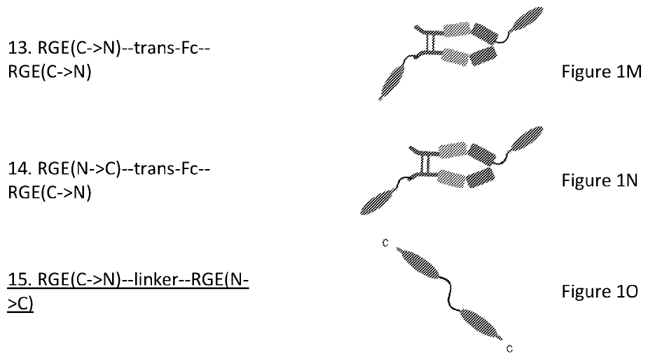

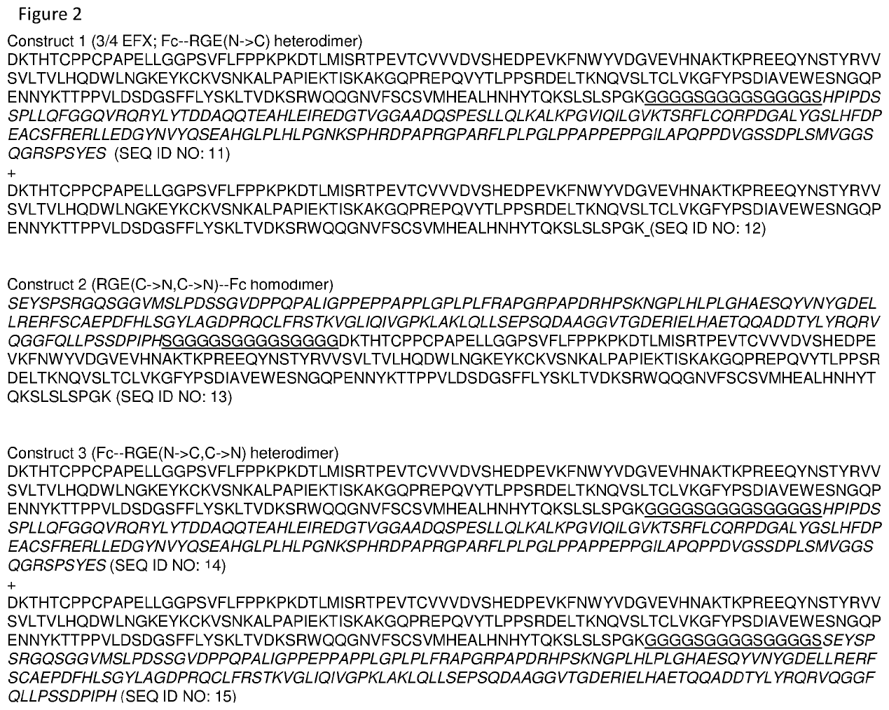

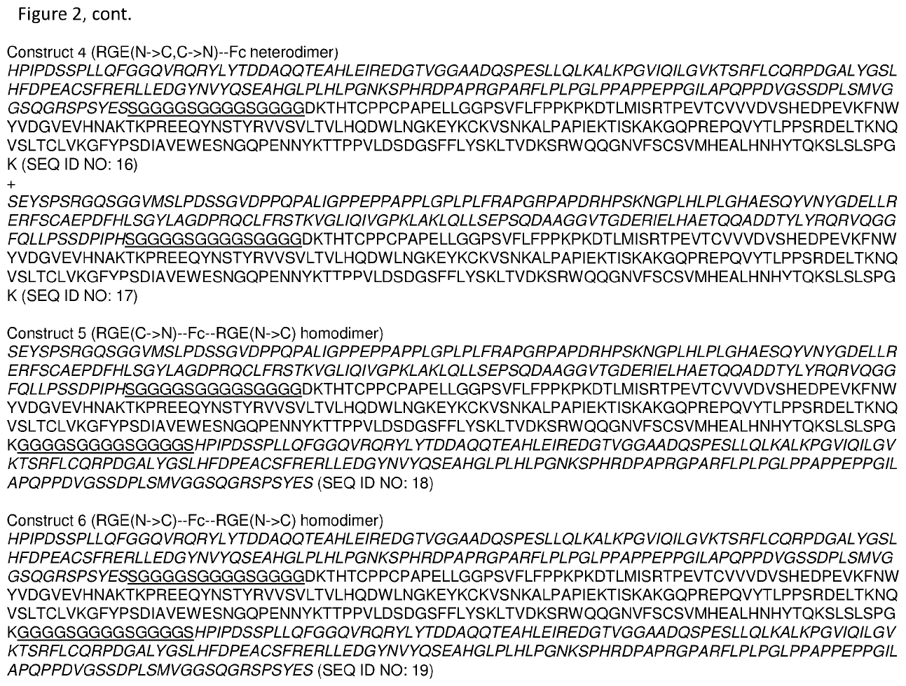

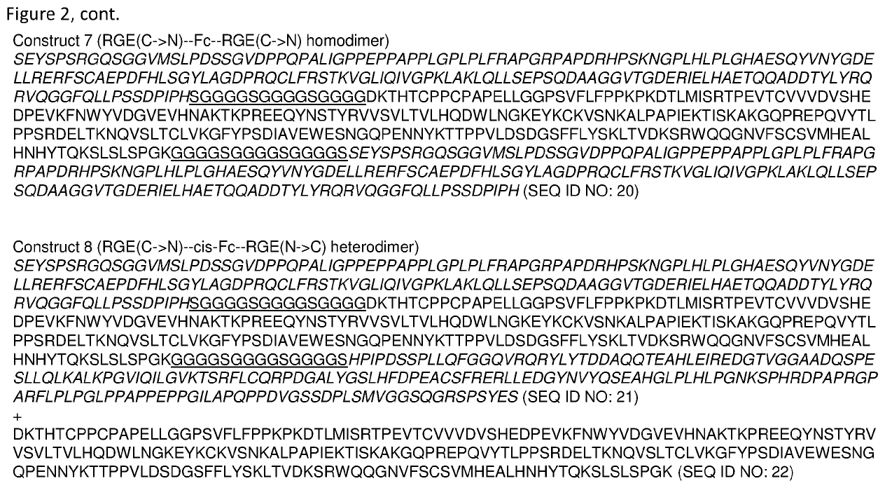

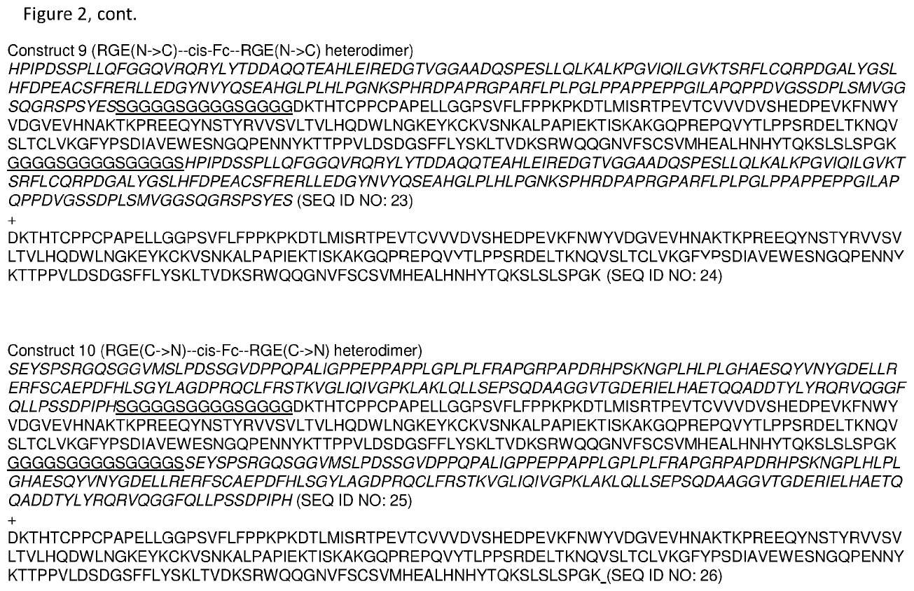

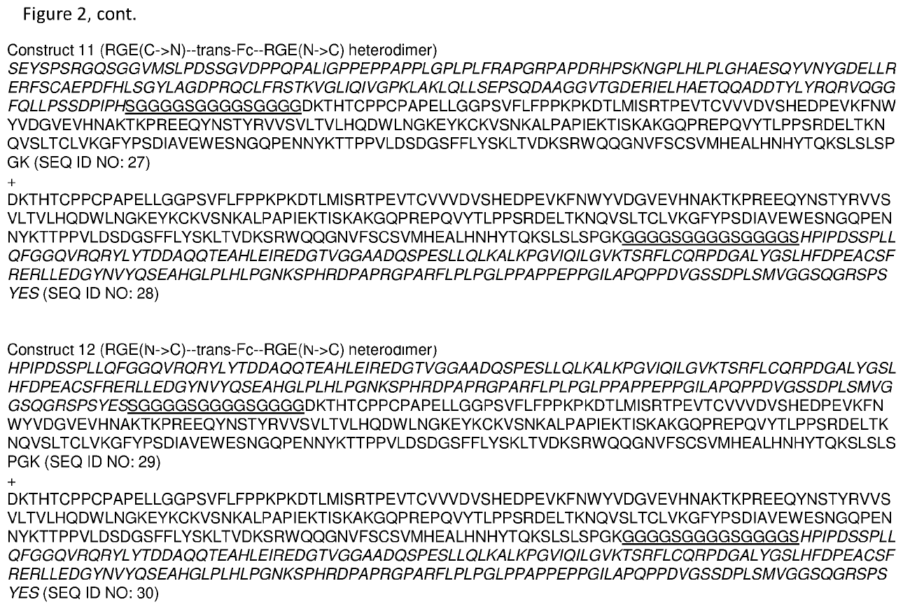

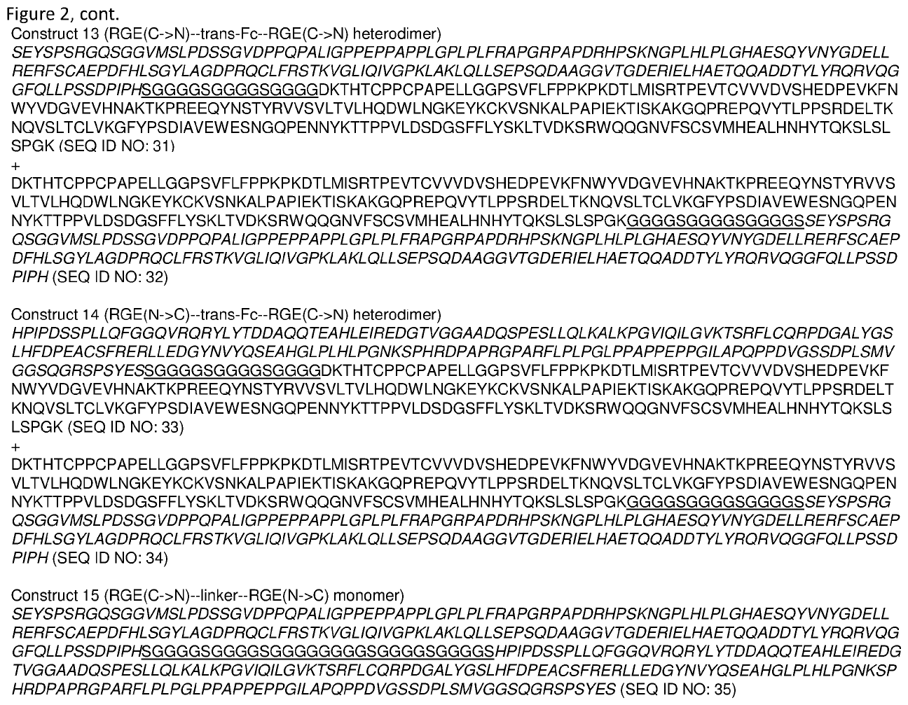

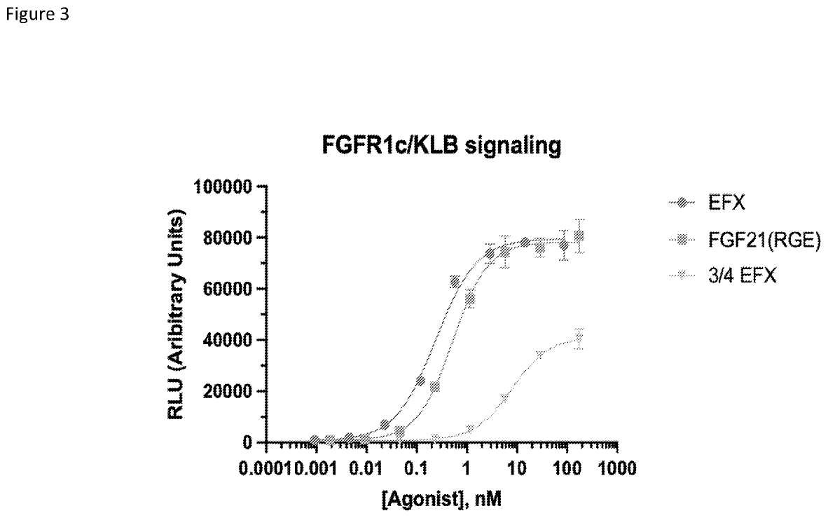

It has been reported that studies using a single-chain FGF21 analog, pegbelfermin, were discontinued because of insufficient efficacy after 24 or more weeks treatment, which included waning pharmacodynamic effect from weeks 4 through 24 of treatment. Sanyal AASLD Poster 2021 LP-40. Studies underlying the present disclosure demonstrate that having at least two, and potentially 3 or more, FGF21 moieties covalently linked intramolecularly offers a molecular configuration better able to better maintain signaling through FGF21's receptors (KLB and FGFR1c), including when levels of receptors present on the cell surface are lower, or when the receptors have mutations resulting in loss of FGF21 signaling. As illustrated in FIG. 3, the addition of an Fc domain to a single-chain FGF21 domain comprising the RGE modifications described above, yielding ¾ EFX (FIG. 1A), reduces potency (right-shift) and extent of agonism (down-shift). However, the addition of a second FGF21 domain to ¾ EFX more than doubles the potency and restores the extent of agonism, suggesting that the two FGF21 domains tethered via a single Fc domain has significantly greater potency than twice the concentration of the ¾ EFX molecule, with only a single FGF21 domain linked to the Fc dimer.

Further, whereas addition of an Fc domain to a single FGF21 (RGE) domain (FIG. 1A; ¾ EFX) reduces the rate of association of the FGF21 analog to the surface of live cells relative to “free” FGF21 (RGE), the addition of a second FGF21 domain to the Fc scaffold both enhances the association rate, and slows the dissociation rate, suggestive of a significantly more stable interaction (>100-fold) between the bivalent FGF21 analog and the surface of the cell compared to either free FGF21 or a single FGF21 domain linked to an Fc domain.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A complex comprising (a) a first monomer comprising an Fc domain fused to two FGF21 domains comprising the amino acid sequence of SEQ ID NO: 2 or SEQ ID NO: 4, which may be the same or different, and (b) a second monomer comprising an Fc domain fused to at least one FGF21 domain comprising the amino acid sequence of SEQ ID NO: 2 or SEQ ID NO: 4, wherein at least two of the FGF21 domains of the complex comprise a free beta-klotho binding region.

2. The complex of claim 1, wherein (a) the first monomer comprises the amino acid sequence of SEQ ID NO: 4 fused to the N-terminus of the Fc domain and the amino acid sequence of SEQ ID NO: 2 fused to the C-terminus of the Fc domain.

3. The complex of claim 2, wherein (b) the second monomer comprises the amino acid sequence of SEQ ID NO: 4 fused to the N-terminus of the Fc domain and the amino acid sequence of SEQ ID NO: 2 fused to the C-terminus of the Fc domain.

4. The complex of claim 2, wherein (b) the second monomer comprises the amino acid sequence of SEQ ID NO: 4 fused to the N-terminus of the Fc domain.

5. The complex of claim 2, wherein (b) the second monomer comprises the amino acid sequence of SEQ ID NO: 2 fused to the N-terminus of the Fc domain.

6. The complex of claim 2, wherein (b) the second monomer comprises the amino acid sequence of SEQ ID NO: 4 fused to the C-terminus of the Fc domain.

7. The complex of claim 2, wherein (b) the second monomer comprises the amino acid sequence of SEQ ID NO: 2 fused to the C-terminus of the Fc domain.

8. The complex of claim 1, wherein (a) the first monomer comprises the amino acid sequence of SEQ ID NO: 4 fused to the N-terminus of the Fc domain and the amino acid sequence of SEQ ID NO: 4 fused to the C-terminus of the Fc domain.

9. The complex of claim 8, wherein (b) the second monomer comprises the amino acid sequence of SEQ ID NO: 4 fused to the N-terminus of the Fc domain and the amino acid sequence of SEQ ID NO: 4 fused to the C-terminus of the Fc domain.

10. The complex of claim 8, wherein (b) the second monomer comprises the amino acid sequence of SEQ ID NO: 4 fused to the N-terminus of the Fc domain.

11. The complex of claim 8, wherein (b) the second monomer comprises the amino acid sequence of SEQ ID NO: 2 fused to the C-terminus of the Fc domain.

12. The complex of claim 1, wherein (a) the first monomer comprises the amino acid sequence of SEQ ID NO: 2 fused to the N-terminus of the Fc domain and the amino acid sequence of SEQ ID NO: 2 fused to the C-terminus of the Fc domain.

13. The complex of claim 12, wherein (b) the second monomer comprises the amino acid sequence of SEQ ID NO: 2 fused to the N-terminus of the Fc domain and the amino acid sequence of SEQ ID NO: 2 fused to the C-terminus of the Fc domain.

14. The complex of claim 12, wherein (b) the second monomer comprises the amino acid sequence of SEQ ID NO: 4 fused to the N-terminus of the Fc domain.

15. The complex of claim 12, wherein (b) the second monomer comprises the amino acid sequence of SEQ ID NO: 2 fused to the C-terminus of the Fc domain.

16. The complex of any one of claims 1-15, wherein one or both Fc domains comprise the amino acid sequence of SEQ ID NO: 7.

17. The complex of any one of claims 1-16, wherein one or more FGF21 domains are fused to an Fc domain via a linker comprising the amino acid sequence of SEQ ID NO: 9 or SEQ ID NO: 10.

18. The complex of claim 1 comprising two monomers comprising the amino acid sequence of SEQ ID NO: 18.

19. The complex of claim 1 comprising two monomers comprising the amino acid sequence of SEQ ID NO: 19.

20. The complex of claim 1 comprising two monomers comprising the amino acid sequence of SEQ ID NO: 20.

21. A peptide comprising an Fc domain fused with an FGF21 domain comprising the amino acid sequence of SEQ ID NO: 4 at the N-terminus and an FGF21 domain comprising the amino acid sequence of SEQ ID NO: 2 at the C-terminus.

22. The peptide of claim 21, wherein the Fc domain comprises the amino acid sequence of SEQ ID NO: 7.

23. The peptide of claim 21 or claim 22, wherein the FGF21 domain comprising SEQ ID NO: 4 is fused to the Fc domain via a linker comprising the amino acid sequence of SEQ ID NO: 10 and the FGF21 domain comprising SEQ ID NO: 2 is fused to the Fc domain via a linker comprising the amino acid sequence of SEQ ID NO: 9.

24. The peptide of claim 21, comprising the amino acid sequence of SEQ ID NO: 21.

25. A complex comprising (a) the peptide of any one of claims 21-24 and (b) a second Fc domain.

26. A complex comprising (a) a first monomer comprising an Fc domain fused at the C-terminus with an FGF21 domain comprising the amino acid sequence of SEQ ID NO: 4 and (b) a second monomer comprising an Fc domain fused at the N-terminus with an FGF21 domain comprising the amino acid sequence of SEQ ID NO: 2.

27. The complex of claim 26, wherein at least one Fc domain comprises the amino acid sequence of SEQ ID NO: 7.

28. The complex of claim 26 or claim 27, wherein the FGF21 domain comprising SEQ ID NO: 4 is fused to the Fc domain of the first monomer via a linker comprising the amino acid sequence of SEQ ID NO: 10 and the FGF21 domain comprising SEQ ID NO: 2 is fused to the Fc domain of the second monomer via a linker comprising the amino acid sequence of SEQ ID NO: 9.

29. The complex of claim 26, wherein the first monomer comprises the amino acid sequence of SEQ ID NO: 27 and the second monomer comprises the amino acid sequence of SEQ ID NO: 28.

30. A peptide comprising the amino acid sequence of SEQ ID NO: 4 fused to the amino acid sequence of SEQ ID NO: 2 via a linker comprising the amino acid sequence of SEQ ID NO: 9 and/or the amino acid sequence of SEQ ID NO: 10.

31. The complex or peptide of any one of claims 1-30, wherein the Fc domain is an IgG1 Fc domain.

32. A nucleic acid comprising a nucleotide sequence encoding the first monomer of any one of claims 1-20.

33. A nucleic acid comprising a nucleotide sequence encoding the second monomer of any one of claims 1-20.

34. An expression vector comprising the nucleic acid of claim 32.

35. An expression vector comprising the nucleic acid of claim 33.

36. A system comprising an expression vector comprising a nucleic acid comprising a nucleotide sequence encoding the first monomer of any one of claims 1-20 and an expression vector comprising a nucleic acid comprising a nucleotide sequence encoding the second monomer of any one of claims 1-20.

37. The system of claim 36, comprising
(a) an expression vector comprising a nucleic acid comprising a nucleotide sequence encoding the first monomer of any one of claims 1-7 and an expression vector comprising a nucleic acid comprising a nucleotide sequence encoding the second monomer of any one of claims 1-7;
(b) an expression vector comprising a nucleic acid comprising a nucleotide sequence encoding the first monomer of any one of claims 8-11 and an expression vector comprising a nucleic acid comprising a nucleotide sequence encoding the second monomer of any one of claims 8-11; or
(c) an expression vector comprising a nucleic acid comprising a nucleotide sequence encoding the first monomer of any one of claims 12-15 and an expression vector comprising a nucleic acid comprising a nucleotide sequence encoding the second monomer of any one of claims 12-15.

38. A host cell comprising the expression vector of claim 34 and/or claim 35.

39. A method of producing a complex, the method comprising culturing a host cell comprising the system of claim 37 and harvesting the complex.

40. A nucleic acid comprising a nucleotide sequence encoding the peptide of any one of claims 21-24 and 30.

41. A nucleic acid comprising a nucleotide sequence encoding the first monomer of any one of claims 26-29 or the second monomer of any one of claims 26-29.

42. An expression vector comprising the nucleic acid of claim 40 or claim 41.

43. A host cell comprising the expression vector of claim 42.

44. A method of producing a complex, the method comprising culturing a host cell comprising an expression vector comprising a nucleic acid comprising a nucleotide sequence encoding the first monomer of any one of claims 26-29 and an expression vector comprising a nucleic acid comprising a nucleotide sequence encoding the second monomer of any one of claims 26-29, and harvesting the complex.

45. A pharmaceutical composition comprising the complex or peptide of any one of claims 1-30.

46. A method of treating nonalcoholic steatohepatitis (NASH) or nonalcoholic fatty liver disease (NAFLD), comprising administering the pharmaceutical composition of claim 45 to a subject in need thereof.

47. A method of reversing NASH with cirrhosis, comprising administering the pharmaceutical composition of claim 45 to a subject in need thereof.

48. A method of treating alcoholic steatohepatitis (ASH), alcoholic liver disease (ALD) or alcoholic fatty liver disease (AFLD), comprising administering the pharmaceutical composition of claim 45 to a subject in need thereof.

49. A method of normalizing liver fat content in a subject in need thereof, comprising administering the pharmaceutical composition of claim 45 to a subject in need thereof.

50. A method of reversing liver cirrhosis or reducing fibrosis associated with NASH, NAFLD, ASH, ALD, or AFLD, comprising administering the pharmaceutical composition of claim 45 to a subject in need thereof.

51. A method of treating type 2 diabetes, comprising administering the pharmaceutical composition of claim 45 to a subject in need thereof.

52. A method of treating obesity, comprising administering the pharmaceutical composition of claim 45 to a subject in need thereof.

53. A method of treating dyslipidemia, comprising administering the pharmaceutical composition of claim 45 to a subject in need thereof.

54. A method of lowering blood glucose, comprising administering the pharmaceutical composition of claim 45 to a subject in need thereof.

55. A method of increasing insulin sensitivity, comprising administering the pharmaceutical composition of claim 45 to a subject in need thereof.

56. A method of reducing uric acid, comprising administering the pharmaceutical composition of claim 45 to a subject in need thereof.

57. A method of treating craving or addiction, comprising administering the pharmaceutical composition of claim 45 to a subject in need thereof.

58. The method of any one of claims 46-57, wherein pharmaceutical composition comprises the complex of any one of claims 18-20.

59. A method of treating metabolic dysfunction-associated steatohepatitis (MASH) or metabolic dysfunction-associated steatotic liver disease (MASLD), comprising administering the pharmaceutical composition of claim 45 to a subject in need thereof.

60. A method of reversing MASH with cirrhosis, comprising administering the pharmaceutical composition of claim 45 to a subject in need thereof.

61. A method of treating metabolic-dysfunction associated alcoholic liver disease (MetALD), comprising administering the pharmaceutical composition of claim 45 to a subject in need thereof.

62. A method of reversing liver cirrhosis or reducing fibrosis associated with MASH, MAFLD, MetALD, comprising administering the pharmaceutical composition of claim 45 to a subject in need thereof.

63. The complex of any of claims 1-17, wherein the first monomer and/or the second monomer comprises two or more tandem FGF21 domains comprising SEQ ID NO: 2 and/or SEQ ID NO: 4 fused to the N-terminus of the Fc domain.

64. The complex or peptide of any one of claims 1-15, 17-21, 23-26, and 28-30, wherein the Fc domain comprises the amino acid sequence of any one of SEQ ID NO: 76-83.


SEQUENCE LISTING (83 sequences)

SEQ ID NO: 1 - protein, 424 aa, synthetic construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 2 - protein, 181 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 3 - protein, 9 aa, synthetic construct
QGRSPSYES

SEQ ID NO: 4 - protein, 181 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 5 - protein, 182 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 6 - protein, 9 aa, synthetic construct
SEYSPSRGQ

SEQ ID NO: 7 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 228 aa, synthetic construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 15 aa, synthetic construct
GGGGSGGGGSGGGGS

SEQ ID NO: 10 - protein, 15 aa, synthetic construct
SGGGGSGGGGSGGGG

SEQ ID NO: 11 - protein, 423 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 12 - protein, 227 aa, synthetic construct; identical to SEQ ID NO: 7

SEQ ID NO: 13 - protein, 423 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 423 aa, synthetic construct; identical to SEQ ID NO: 11

SEQ ID NO: 15 - protein, 423 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 16 - protein, 423 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYESSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 423 aa, synthetic construct; identical to SEQ ID NO: 13

SEQ ID NO: 18 - protein, 619 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 19 - protein, 619 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYESSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 20 - protein, 619 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 21 - protein, 619 aa, synthetic construct; identical to SEQ ID NO: 18

SEQ ID NO: 22 - protein, 227 aa, synthetic construct; identical to SEQ ID NO: 7

SEQ ID NO: 23 - protein, 619 aa, synthetic construct; identical to SEQ ID NO: 19

SEQ ID NO: 24 - protein, 227 aa, synthetic construct; identical to SEQ ID NO: 7

SEQ ID NO: 25 - protein, 619 aa, synthetic construct; identical to SEQ ID NO: 20

SEQ ID NO: 26 - protein, 227 aa, synthetic construct; identical to SEQ ID NO: 7

SEQ ID NO: 27 - protein, 423 aa, synthetic construct; identical to SEQ ID NO: 13

SEQ ID NO: 28 - protein, 423 aa, synthetic construct; identical to SEQ ID NO: 11

SEQ ID NO: 29 - protein, 423 aa, synthetic construct; identical to SEQ ID NO: 16

SEQ ID NO: 30 - protein, 423 aa, synthetic construct; identical to SEQ ID NO: 11

SEQ ID NO: 31 - protein, 423 aa, synthetic construct; identical to SEQ ID NO: 13

SEQ ID NO: 32 - protein, 423 aa, synthetic construct; identical to SEQ ID NO: 15

SEQ ID NO: 33 - protein, 423 aa, synthetic construct; identical to SEQ ID NO: 16

SEQ ID NO: 34 - protein, 423 aa, synthetic construct; identical to SEQ ID NO: 15

SEQ ID NO: 35 - protein, 392 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 36 - protein, 424 aa, synthetic construct; identical to SEQ ID NO: 1

SEQ ID NO: 37 - protein, 243 aa, synthetic construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGS

SEQ ID NO: 38 - protein, 424 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 424 aa, synthetic construct; identical to SEQ ID NO: 1

SEQ ID NO: 40 - protein, 424 aa, synthetic construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 41 - protein, 424 aa, synthetic construct
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYESSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 424 aa, synthetic construct; identical to SEQ ID NO: 38

SEQ ID NO: 43 - protein, 620 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 44 - protein, 620 aa, synthetic construct
MHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYESSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 45 - protein, 620 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 46 - protein, 620 aa, synthetic construct; identical to SEQ ID NO: 43

SEQ ID NO: 47 - protein, 228 aa, synthetic construct; identical to SEQ ID NO: 8

SEQ ID NO: 48 - protein, 620 aa, synthetic construct; identical to SEQ ID NO: 44

SEQ ID NO: 49 - protein, 228 aa, synthetic construct; identical to SEQ ID NO: 8

SEQ ID NO: 50 - protein, 620 aa, synthetic construct; identical to SEQ ID NO: 45

SEQ ID NO: 51 - protein, 243 aa, synthetic construct; identical to SEQ ID NO: 37

SEQ ID NO: 52 - protein, 424 aa, synthetic construct; identical to SEQ ID NO: 38

SEQ ID NO: 53 - protein, 424 aa, synthetic construct; identical to SEQ ID NO: 1

SEQ ID NO: 54 - protein, 424 aa, synthetic construct; identical to SEQ ID NO: 41

SEQ ID NO: 55 - protein, 424 aa, synthetic construct; identical to SEQ ID NO: 1

SEQ ID NO: 56 - protein, 424 aa, synthetic construct; identical to SEQ ID NO: 38

SEQ ID NO: 57 - protein, 424 aa, synthetic construct; identical to SEQ ID NO: 40

SEQ ID NO: 58 - protein, 424 aa, synthetic construct; identical to SEQ ID NO: 41

SEQ ID NO: 59 - protein, 424 aa, synthetic construct; identical to SEQ ID NO: 40

SEQ ID NO: 60 - protein, 393 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 61 - protein, 393 aa, synthetic construct
SEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGXGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 62 - protein, 394 aa, synthetic construct
MSEYSPSRGQSGGVMSLPDSSGVDPPQPALIGPPEPPAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPHSGGGGSGGGGSGGGGXGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 63 - protein, 181 aa, synthetic construct
  MOD_RES 146: note = "P" or "L"
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAXPEPPGILAPQPPDVGSSDPLSMVGGSQGRSPSYES

SEQ ID NO: 64 - protein, 181 aa, synthetic construct
  MOD_RES 36: note = "P" or "L"
SEYSPSRGQSGGVMSLPDSSGVDPPQPALIGPPEPXAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 65 - protein, 181 aa, synthetic construct
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 66 - protein, 181 aa, synthetic construct
SEYSPSRGQSGGVMSLPDSSGVDPPQPALIGPPEPLAPPLGPLPLFRAPGRPAPDRHPSKNGPLHLPLGHAESQYVNYGDELLRERFSCAEPDFHLSGYLAGDPRQCLFRSTKVGLIQIVGPKLAKLQLLSEPSQDAAGGVTGDERIELHAETQQADDTYLYRQRVQGGFQLLPSSDPIPH

SEQ ID NO: 67 - protein, 5 aa, synthetic construct
GGGGS

SEQ ID NO: 68 - protein, 5 aa, synthetic construct
SGGGG

SEQ ID NO: 69 - protein, 5 aa, synthetic construct
GSSSS

SEQ ID NO: 70 - protein, 4 aa, synthetic construct
GGGS

SEQ ID NO: 71 - protein, 10 aa, synthetic construct
GGGGSGGGGS

SEQ ID NO: 72 - protein, 15 aa, synthetic construct
GGGGSGGGGSGGGGS

SEQ ID NO: 73 - protein, 20 aa, synthetic construct
GGGGSGGGGSGGGGSGGGGS

SEQ ID NO: 74 - protein, 25 aa, synthetic construct
GGGGSGGGGSGGGGSGGGGSGGGGS

SEQ ID NO: 75 - protein, 619 aa, synthetic construct; identical to SEQ ID NO: 19
  REPEAT 1..196: note = tandem

SEQ ID NO: 76 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 227 aa, synthetic construct
DKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 78 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 79 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - protein, 228 aa, synthetic construct
MDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 228 aa, synthetic construct
MDKTHTCPPCPAPELLGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 82 - protein, 228 aa, synthetic construct
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 83 - protein, 228 aa, synthetic construct
MDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK